(19) United States
(12) Patent Application Publication
(10) Pub. No.: US 2014/0343692 A1
(43) Pub. Date: Nov. 20, 2014

(54) COVERING FOR A LIMB PROSTHESIS, LIMB PROSTHESIS, AND MANUFACTURING METHOD THEREOF

(71) Applicant: AQUALEG, Nantes (FR)

(72) Inventor: Frédéric Rauch, Nantes (FR)

(73) Assignee: AQUALEG, Nantes (FR)

(21) Appl. No.: 14/357,232

(22) PCT Filed: Nov. 6, 2012
(86) PCT No.: PCT/FR2012/052560

§ 371 Date: May 9, 2014

(30) Foreign Application Priority Data
Nov. 21, 2011 (FR) 1160571

(51) Int. Cl. A61F 2/78; A61F 2/76; A61F 2/60

(52) U.S. Cl. CPC A61F 2/78; A61F 2/60; A61F 2/76; A61F 2002/5001
USPC 623/27; 156/324

(57) ABSTRACT

A covering (1) intended to cover the body (10) of a limb prosthesis, the covering (1) being a tubular covering including superposed layers including at least one silicone layer (31). The covering has at least one silicone layer (2), which is a transparent or translucent silicone layer that forms the external layer of the covering (1). The covering additionally includes at least two layers (41, 42, 43, 44) of dyed elastic fabric, preferably in different shades, at least the closest layer of elastic fabric (41) of the external layer (2) being a light-permeable layer.

The present invention relates to a covering for a limb prosthesis body, intended to dress said prosthesis body, to a limb prosthesis whose prosthesis body is covered by such a covering, and to a method for manufacturing such a covering.

It relates more particularly to a tubular covering comprising a superposition of layers, of which at least one is a layer of silicone.

The cosmetic coverings for prosthesis bodies, also called skins or envelopes, are well known to those skilled in this art and are intended to protect the prosthesis body and to give it an esthetic appearance more pleasing to the eye, as is illustrated in the patent GB 2 067 074, in which such a covering is produced with the aid of two superposed layers of silicone, the external layer being a transparent layer, which covers an internal layer of silicone that is tinted the color of skin all the way through. This design has a number of disadvantages. The fact that the silicone is tinted all the way through gives the finished product an appearance that is often remote from the natural appearance of human skin. In addition, the flexion of the prosthesis results in the silicone being weakened and eventually tearing. If the silicone is too rigid, it also has the disadvantage of impeding flexion and, consequently, the patient's gait.

It is therefore an aim of the present invention to make available a covering of the aforementioned type whose design permits resemblance to human skin without adversely affecting the mechanical strength of said covering and without impeding flexion.

To this end, the subject matter of the invention is a covering for a limb prosthesis body, intended to dress said prosthesis body, said covering being a tubular covering comprising a superposition of layers, of which at least one is a layer of silicone, characterized in that said covering, of which the or at least one layer of silicone is a layer of transparent or translucent silicone that forms the external layer of the covering, additionally comprises at least two layers of tinted elastic fabric, preferably of different tints, of which at least the layer of elastic fabric closest to the external layer is a layer permeable to light.

The superposition of a layer of transparent or translucent silicone and of a layer of elastic fabric is able to give said layer of silicone its mechanical strength. The superposition of two layers of elastic fabric makes it possible to act on the permeability to light and on the resulting transparency of at least one of the layers of fabric in order to obtain, by superposition of the layers of fabric, which are generally of different tints, a large number of combinations and a more realistic effect close to the appearance of human skin. Thus, by superposing several color tints, the aim is to obtain, by a transparency effect, a final color or tint close to the desired skin color. It should be noted that the term “tinted” is an equivalent of the term “colored”. A tinted elastic fabric is therefore a colored fabric. Two fabrics of different tint are fabrics of different color. A tinted elastic fabric can be tinted throughout the body thereof and/or at the surface.

Preferably, the covering comprises at least two layers of silicone, between which the two, or at least two, layers of tinted elastic fabric are disposed. This combination is particularly advantageous when the covering is intended for a prosthesis that is designed to go in water, so as to obtain water-tightness and a sufficient mechanical strength of said prosthesis without adversely affecting the appearance of the latter.

Generally, the layer of silicone, unlike the external layer of silicone, is an opaque screen-forming layer.

Preferably, the superposition of layers comprises, from the outside toward the inside of the covering, at least one external layer of transparent or translucent silicone, two layers of elastic fabric, a layer of silicone, and a layer of elastic fabric.

Independently of the number of intermediate layers, the external layer is a layer of transparent or translucent silicone, whereas the internal layer of the covering is a layer of elastic fabric.

The covering preferably comprises, interposed between two layers of tinted elastic fabric, at least one non-continuous layer of paint, preferably in the form of a mist. The presence of this mist makes it possible to give said covering the irregular appearance of the skin.

The elastic fabric of at least one of the layers of elastic fabric is preferably a mesh fabric comprising at least a polyamide.

Generally, at least one or preferably each layer of elastic fabric has a linear density of the yarn in the range of 1×10⁻³ to 3.33×10⁻³ g/m, preferably 1.33×10⁻³ to 2.77×10⁻³ g/m.

Preferably, with said covering being a covering for the body of a femoral prosthesis, said covering comprises, in the area situated above the knee, a fold for reserve stretching.

The invention also relates to a limb prosthesis comprising at least one prosthesis body covered by a covering able to be engaged on said body, characterized in that said covering is of the aforementioned type.

The invention also relates to a method for manufacture of a covering for a limb prosthesis body of the aforementioned type, intended to dress said body, characterized in that said method comprises at least:
a) a step during which at least one layer of elastic fabric in the form of a sleeve is engaged on a positive mold of the limb to be restored,
b) a step during which the layer of elastic fabric last engaged is covered with a layer of silicone,

and in that step a) is repeated at least twice and step b) at least once in order to obtain a superposition of layers comprising, from the outside toward the inside of the covering, at least one external layer of transparent or translucent silicone, at least two layers of elastic fabric, a layer of silicone, and at least one layer of elastic fabric.

The invention will become clear on reading the following description of illustrative embodiments and by referring to the attached drawings, in which:

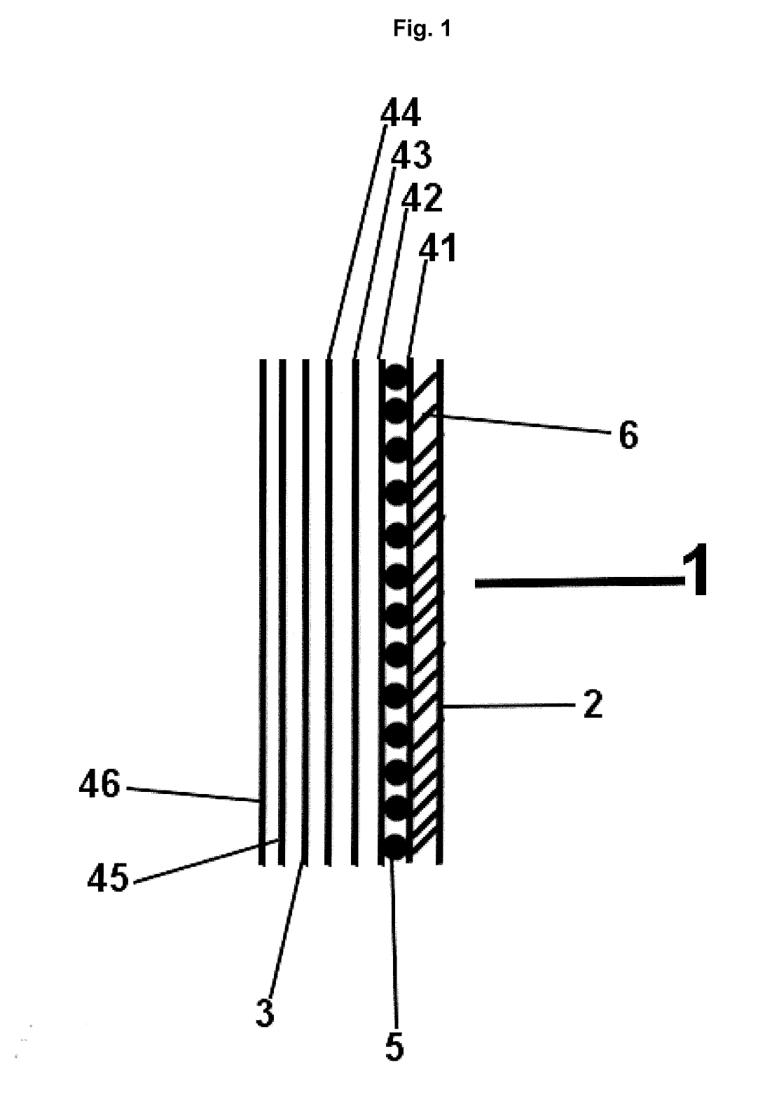

FIG. 1 shows a schematic cross-sectional view through the thickness of a covering;

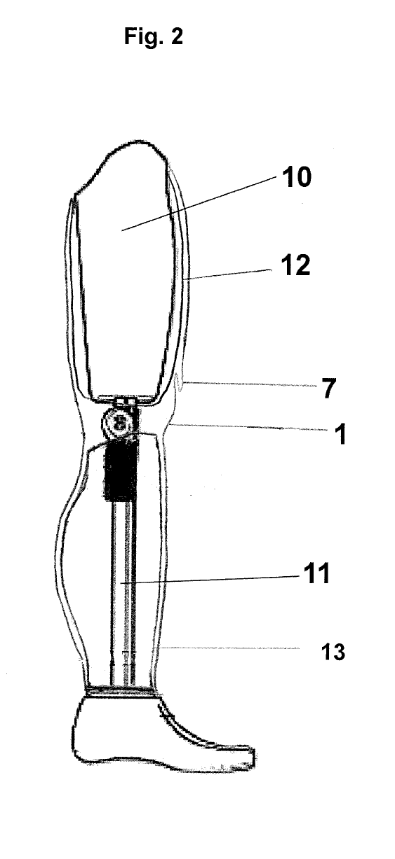

FIG. 2 shows a phantom view of a femoral prosthesis equipped with a covering according to the invention.

As has been mentioned above, the covering 1 according to the invention is more particularly intended to cover a limb prosthesis body 10, in order to dress said prosthesis body 10. In the example shown in FIG. 2, the prosthesis is a femoral prosthesis, of which the prosthesis body 10 comprises two shells, one being an upper shell designated by 12, the other being a lower shell designated by 13. The lower shell 13 and the upper shell 12 cover an endoskeleton structure 11. This endoskeleton structure 11 here comprises a shaft covered by the lower shell 13, this shaft being continued beyond the lower shell via a joint that connects said shells to each other. The covering 1 according to the invention is engaged on the prosthesis body 10. This covering is presented, like a sleeve, in the form of a tubular covering open at each of its ends. This covering is formed by a plurality of superposed layers. The external layer 2 of this covering is a layer of transparent or translucent silicone. A layer of transparent silicone is preferred. This covering 1 additionally comprises at least two layers 41, 42 of tinted elastic fabric, generally of different tints, of which at least the layer 41 of elastic fabric closest to the external layer 2 is a layer permeable to light. Each layer of elastic fabric is also presented in the form of a tubular body or sleeve. Generally, the base tint of at least one of the layers of elastic fabric corresponds to the skin color of the person wearing the prosthesis.

In the example shown, the covering 1 comprises two layers 2, 3 of silicone, between which layers 41, 42, 43, 44 of tinted elastic fabric are disposed. The presence of these two layers of silicone, which sandwich the layers of elastic fabric, makes it possible to avoid impregnation of the fabrics by capillarity, for example in the case of prolonged activity in water.

Preferably, the internal layer 3 of silicone is an opaque layer forming a screen for the image formed by the superposition of the layers of elastic fabric that are disposed between said layers 2, 3 of silicone. When the second layer 3 of silicone is present, as in FIG. 2, this layer 3 of silicone is lined internally by at least one layer of elastic fabric. In the example shown here, the covering 1 has, to the inside of the layer 3 of silicone, two layers 45, 46 of elastic fabric of any given tint. By contrast, the tint of the succession of layers of elastic fabric extending behind the external layer of transparent silicone gives said covering its color. Each layer 41, 42, 43, 44 of elastic fabric makes its contribution to the final tint or color of said covering, which is generally close to the color of the skin of the patient. The tints of each layer of elastic fabric cannot therefore be chosen at random, likewise the order, the number and the density of the layers of elastic fabric, which depend on the desired esthetic result. Generally, each layer of elastic fabric is a mesh fabric comprising at least a polyamide. By way of example, it is possible to use an elastic fabric made of polyamide, or of polyamide and elastane, or of polyamide, polyurethane and elastane. Other combinations are of course conceivable. The elastic fabrics also called stockings or tights sold by the company Calzificio Mura S.P.A. under the name Myto provide complete satisfaction.

Preferably, each layer 41 to 46 of elastic fabric has a linear density of the yarn in the range of 1×10⁻³ to 3.33×10⁻³ g/m, preferably 1.33×10⁻³ to 2.77×10⁻³ g/m. Expressed in denier, the linear density is between 9 and 30 deniers, preferably between 12 and 25 deniers. The tint of each layer of fabric can vary within a wide range of colors. Thus, it is possible to superpose a 15-denier fabric composed of 90% polyamide and 10% elastane, with a tint expressed as Pantone 4665C, and a 20-denier fabric of 100% polyamide whose Pantone tint bears reference 4655 C. To increase the similarity to the appearance of human skin, the covering 1 preferably comprises, between two layers 41, 42 of tinted elastic fabric, at least one non-continuous layer 5 of paint, preferably in the form of a mist. Again, the number of layers of paint and their arrangement in the superposition of layers can influence the final appearance of the covering. Generally, during the manufacture of the covering, the paint is sprayed onto the layer of elastic fabric in order to form a light-permeable layer at the surface of said layer of elastic fabric. Finally, at least one layer 6 of artificial hair can be provided between two layers 41, 42 of elastic fabric or between the external layer 2 of silicone and a layer of elastic fabric. These artificial hairs can adhere to the layer of elastic fabric by static electricity or by adhesive bonding.

To perfect the covering 1, in the case where this covering is a covering for the body of a femoral prosthesis, said covering 1 comprises, in the area situated above the knee, a fold 7 for reserve stretching.

A covering for a prosthesis body in accordance with FIG. 2 is manufactured as follows. In a first step, at least one layer 46 of elastic fabric in the form of a sleeve is engaged on a positive mold of the limb that is to be restored. A second layer 45 of elastic fabric, again in the form of a sleeve, is engaged on this same positive mold so as to superpose the layer 46 of elastic fabric. This assembly is then covered by a layer 3 of silicone. The silicone can be applied by spraying or manual coating. Layers of elastic fabric are then superposed by each being engaged on the positive mold by virtue of their presentation in the form of a sleeve. Thus, layers of elastic fabric designated by reference signs 44, 43, 42 and 41 in FIG. 1 are successively engaged. Each time, the layers of elastic fabric used can be of different linear density, tint or composition. A paint is sprayed on between the layers 42 and 41. The whole assembly is then covered with an external layer 2 of transparent silicone. Again, this application of the silicone can be carried out by spraying or manual coating. A layer 6 of artificial hair can also be added between the last layer of elastic fabric 41 and the external layer 2 of transparent silicone. Once the silicone has hardened at ambient temperature, the manufacture of the covering is complete.

1. A covering (1) for a limb prosthesis body (10), intended to dress said prosthesis body (10), said covering (1) being a tubular covering comprising a superposition of layers, of which at least one is a layer (2) of silicone, characterized in that said covering (1), of which the or at least one layer (2) of silicone is a layer of transparent or translucent silicone that forms the external layer of the covering (1), additionally comprises at least two layers (41, 42, 43, 44) of tinted elastic fabric, preferably of different tints, of which at least the layer (41) of elastic fabric closest to the external layer (2) is a layer that is permeable to light.

2. The covering (1) as claimed in claim 1, characterized in that the covering (1) comprises at least two layers (2, 3) of silicone, between which the two, or at least two, layers (41, 42, 43, 44) of tinted elastic fabric are disposed.

3. The covering (1) as claimed in claim 2, characterized in that the layer (3) of silicone, unlike the external layer (2) of silicone, is an opaque screen-forming layer.

4. The covering (1) as claimed in claim 1, characterized in that the superposition of layers comprises, from the outside toward the inside of the covering (1), at least one external layer (2) of transparent or translucent silicone, at least two layers (41, 42) of elastic fabric, a layer (3) of silicone, and at least one layer (45) of elastic fabric.

5. The covering (1) as claimed in claim 1, characterized in that it comprises, interposed between two layers (41, 42) of tinted elastic fabric, at least one non-continuous layer (5) of paint, preferably in the form of a mist.

6. The covering (1) as claimed in claim 1, characterized in that it comprises, between two layers (41, 42) of elastic fabric or between the external layer (2) of silicone and a layer (41) of elastic fabric, at least one layer (6) of artificial hair.

7. The covering (1) as claimed in claim 1, characterized in that the elastic fabric of at least one of the layers (41, 42, 43, 44, 45, 46) of elastic fabric is a mesh fabric comprising at least a polyamide.

8. The covering (1) as claimed in claim 1, characterized in that at least one or preferably each layer (41 to 46) of elastic fabric has a linear density of the yarn in the range of 1×10⁻³ to 3.33×10⁻³ g/m, preferably 1.33×10⁻³ to 2.77×10⁻³ g/m.

9. The covering (1) as claimed in claim 1, characterized in that, with said covering (1) being a covering for the body of a femoral prosthesis, said covering (1) comprises, in the area situated above the knee, a fold (7) for reserve stretching.

10. A limb prosthesis comprising at least one prosthesis body (10) covered by a covering (1) able to be engaged on said body (10), characterized in that said covering (1) is as claimed in claim 1.

11. A method for manufacture of a covering (1) for a limb prosthesis body (10) as claimed in claim 1, intended to dress said body (10), characterized in that said method comprises at least:
a) a step during which at least one layer (45, 46) of elastic fabric in the form of a sleeve is engaged on a positive mold of the limb to be restored,
b) a step during which the layer (45) of elastic fabric last engaged is covered with a layer (3) of silicone,
and in that step a) is repeated at least twice and step b) at least once in order to obtain a superposition of layers comprising, from the outside toward the inside of the covering (1), at least one external layer (2) of transparent or translucent silicone, at least two layers (41, 42, 43, 44) of elastic fabric, a layer (3) of silicone, and at least one layer (45, 46) of elastic fabric.
